(19) United States
(12) Patent Application Publication
(10) Pub. No.: US 2018/0039732 A1
(43) Pub. Date: Feb. 8, 2018

(54) DASATINIB RESPONSE PREDICTION MODELS AND METHODS THEREFOR

(71) Applicant: NantOmics, LLC, Culver City, CA (US)

(72) Inventors: Christopher W. Szeto, Scotts Valley, CA (US); Stephen Charles Benz, Santa Cruz, CA (US); Charles Joseph Vaske, Santa Cruz, CA (US)

(21) Appl. No.: 15/668,616

(22) Filed: Aug. 3, 2017

Related U.S. Application Data
(60) Provisional application No. 62/370,657, filed on Aug. 3, 2016.

(51) Int. Cl. G06F 19/24; A61K 45/06; G01N 33/50

(52) U.S. Cl. CPC G06F 19/24; G01N 33/50; A61K 45/06; G06N 3/00

(57) ABSTRACT

Contemplated systems and methods employ a priori known cell line genomics and drug response data to build a library of response predictors across multiple and distinct cell types and drugs. Statistical analysis of selected response predictors is then employed to identify a drug with a response predictor that has significant gain in prediction power relative to other drugs. Entity coefficients of the so identified response predictor are then applied to the output of a pathway model that was based on an actual patient's omic signature.

This application claims priority to US provisional application with the Ser. No. 62/370,657, filed 3 Aug. 2016.

### FIELD OF THE INVENTION

The field of the invention is systems and methods of predicting drug responses of a patient to a drug based on pathway model information that is further processed using entity coefficients of a (preferably high-accuracy gain) response predictor.

### BACKGROUND

Various systems and methods of computational modeling of pathways are known in the art. For example, some algorithms (e.g., GSEA, SPIA, and PathOlogist) are capable of successfully identifying altered pathways of interest using pathways curated from literature. Still further tools have constructed causal graphs from curated interactions in literature and have used these graphs to explain expression profiles. Algorithms such as ARACNE, MINDy and CONEXIC take in gene transcriptional information (and copy-number, in the case of CONEXIC) to so identify likely transcriptional drivers across a set of cancer samples. However, these tools do not attempt to group different drivers into functional networks identifying singular targets of interest. Some newer pathway algorithms such as NetBox and Mutual Exclusivity Modules in Cancer (MEMo) attempt to solve the problem of data integration in cancer to thereby identify networks across multiple data types that are key to the oncogenic potential of samples.

While such tools allow for at least some limited integration across pathways to find a network, they generally fail to provide regulatory information and association of such regulatory information with one or more physiological effects in the relevant pathways or network of pathways. In an attempt to improve performance, GIENA looks for dysregulated gene interactions within a single biological pathway but does not take into account the topology of the pathway or prior knowledge about the direction or nature of the interactions. Moreover, due to the relative incomplete nature of these modeling systems, predictive analysis is often impossible, especially where interactions of multiple pathways and/or pathway elements are under investigation.

More recently, improved systems and methods have been described to obtain in silico pathway models of in vivo pathways, and exemplary systems and methods are described in WO 2011/139345 and WO 2013/062505. Further refinement of such models was provided in WO 2014/059036 (collectively referred to herein as “PARADIGM”) disclosing methods to help identify cross-correlations among different pathway elements and pathways. While such models provide valuable insights, for example, into interconnectivities of various signaling pathways and the flow of signals through various pathways, numerous aspects of using such modeling have not been appreciated or even recognized.

All publications and patent applications herein are incorporated by reference to the same extent as if each individual publication or patent application were specifically and individually indicated to be incorporated by reference. Where a definition or use of a term in an incorporated reference is inconsistent or contrary to the definition of that term provided herein, the definition of that term provided herein applies and the definition of that term in the reference does not apply.

Still further progress has been made using insights form PARADIGM as is described in WO 2014/193982. Here, multiple models are obtained from a machine learning system that receives multiple distinct data sets and identifies a determinant pathway element in the distinct data sets that is associated with a status (e.g., sensitive or resistant) of a treatment parameter (e.g., treatment with a drug) of the diseased cells. Such system advantageously provides insight into potential treatment modalities. However, the very large number of potentially valid models obtained from the machine learning system will render simple forecast of treatment outcome difficult.

On the other hand, as described in US 2004/0193019, discriminant analysis-based pattern recognition was employed to generate a model that correlated certain biological profile information with treatment outcome information. The prediction model is then used to rank possible responses to treatment. While such methods may help assess likely outcomes based on patient-specific profile information, analysis is typically biased by the parameters used in the discriminant analysis. Moreover, such analysis only takes into account historical data of corresponding drugs and disease conditions and so limits discovery of drugs known to be effective only in other non-related disease conditions. In addition, availability of the historical data of corresponding drugs and disease conditions tends to further limit usefulness of such methods.

Consequently, it should be appreciated that most, if not all in silico prediction systems and methods are either based on known correlations of disturbances in selected pathway activities with treatment options (e.g., identification of over-activity of a particular kinase activity and likely responsiveness to a particular kinase inhibitor), or empirical in vitro data from non-patient sources. Still further, where machine learning is used to identify patterns, inherent biases of the learning systems tend to skew output in a manner that is not necessarily consistent with the patient's particular situation.

Therefore, even though various systems and methods for prediction of specific drug response are known in the art, there remains a need for systems and methods that allow for simple and robust treatment prediction for a drug with high confidence, and that also allow prediction of the treatment response in a patient specific manner.

### SUMMARY OF THE INVENTION

The inventive subject matter is directed to various devices, systems, and methods in which multiple a priori known cell line genomics and drug-response data are used to build a large number of response predictors having plurality of entity coefficients. Entity coefficients of the best performing response predictor(s) are then used to modify the output of a pathway model to so predict a treatment outcome. Advantageously, such systems and methods are able to integrate multiple pathway elements and interconnections, can be based on patient data, and avoid analytic bias due to use of a single preselected model.

In one aspect of the inventive subject matter, the inventors contemplate a method of processing a plurality of response predictors that includes a step of providing a plurality of response predictors, wherein each of the response predictors is associated with a drug and has a plurality of pathway elements and associated entity coefficients. In another step, an accuracy gain metric is calculated for each of the response predictors relative to a corresponding null model to select a single response predictor, and at least a subset of pathway elements and associated entity coefficients of the selected response predictor and a pathway model output of a patient tumor are used to calculate a score (e.g., sensitivity score with respect to treatment with the drug). Most typically, corresponding null models are calculated using randomly chosen datasets not used in calculation of the response predictors for which the null models are created.

Most typically, the plurality of response predictors is at least 1,000, or at least 10,000, or at least 100,000 response predictors. It is further generally contemplated that the pathway element for the entity coefficient is a regulatory RNA, an immune signaling component, a cell differentiation factor, a cell proliferation factor, an apoptosis signaling component, an angiogenesis factor, and/o a cell cycle checkpoint component.

With respect to the accuracy gain metric it is generally contemplated that the accuracy gain may be determined using accuracy values, accuracy gains, performance metrics, an area under curve metric, an R² value, a p-value metric, a silhouette coefficient, or a confusion matrix. Moreover, it is generally contemplated that the plurality of response predictors are established using at least two, or at least four, or at least six, or at least ten different machine learning classifiers, and suitable machine learning classifiers include a linear kernel support vector machine, a first or second order polynomial kernel support vector machine, a ridge regression, an elastic net algorithm, a sequential minimal optimization algorithm, a random forest algorithm, a naive Bayes algorithm, and a NMF predictor algorithm.

The subset of pathway elements and associated entity coefficients will typically comprise between one and 50 entity coefficients, and it is further contemplated that the pathway model output of the patient tumor comprises pathway elements that are the same as the subset of pathway elements in the selected response predictor.

Therefore, and viewed form a different perspective, the inventors also contemplate a method of using an output of a pathway model of a tumor in a patient for prediction of a treatment outcome of the patient using a drug (e.g., chemotherapeutic drug). Most typically, such method will include a step of using a plurality entity coefficients of pathway elements in a high-accuracy gain response predictor for a drug as factors for output values of corresponding pathway elements in the pathway model of the tumor to predict a treatment outcome score for the patient using the drug. Preferably, the pathway model of the tumor is calculated using omics data of the patient and comprises a plurality of pathway elements and associated output values, and it is further preferred that the high-accuracy gain response predictor has a predetermined minimum accuracy gain relative to a corresponding null model. Additionally, it is preferred in such method that the high-accuracy gain response predictor is selected from a plurality of response predictors, wherein each of the response predictors is associated with the drug.

In typical aspects of such method, the plurality of entity coefficients is between one and 50 entity coefficients of the high-accuracy gain response predictor, and/or the plurality of entity coefficients is a subset of entity coefficients and comprises the top tertile of all entity coefficients of the high-accuracy gain response predictor. While not limiting the inventive subject matter, it is typically preferred that the pathway model is a probabilistic pathway model, and especially PARADIGM.

The predetermined minimum accuracy gain in such contemplated method is at least 50% over the null model, wherein the null model is preferably calculated using randomly chosen datasets not used in calculation of the high-accuracy gain response predictor for which the null model is created. Moreover, it is contemplated that the plurality of response predictors may be relatively large and thus may be at least 1,000, or at least 10,000, or at least 100,000 response predictors, which are most typically established using at least two different machine learning classifiers (e.g., linear kernel support vector machine, first or second order polynomial kernel support vector machine, ridge regression, elastic net algorithm, sequential minimal optimization algorithm, random forest algorithm, naive Bayes algorithm, NMF predictor algorithm, etc.).

Therefore, in one exemplary aspect of the inventive subject matter, a method of predicting a treatment outcome for treatment of a tumor of a patient with dasatinib is contemplated. Such method will preferably include the steps of (a) obtaining omics data of the tumor of the patient, (b) calculating by a pathway analysis engine that uses a pathway model and the omics data, a pathway model output for the tumor, wherein the pathway output comprises a plurality of pathway elements and associated activity values, and (c) applying a plurality of entity coefficients of respective pathway entities as factors to the activity values of corresponding pathway elements of the pathway model output to thereby predict the treatment outcome for the patient. The pathway entities and respective entity coefficients for such methods are preferably are selected from the group consisting of MIR34A_(miRNA): −0.10545895; ETS1: −0.094264817; 5_8_S_rRNA_(rna): 0.086044958; CEBPB_(dimer)_(complex): 0.067691407; FOSL1: −0.067263561; CEBPB: 0.066698569; JUN/FOS_(complex): −0.064549881; Fra1/JUN_(complex): −0.060403293; FOXA2: 0.059755319; FOS: −0.059560833; E2F1: −0.050992273; AP1_(complex): −0.049823492; anoikis_(abstract): −0.04853399; FOXA1: 0.035994367; dNp63a_(tetramer)_(complex): −0.033478521; TP63: −0.02956134; MYC: 0.026847479; TP63-2: −0.026423542; E2F-1/DP-1_(complex): −0.023462081; MYB: 0.022211938; TAp63g_(tetramer)_(complex): 0.019789929; HIF1A/ARNT_(complex): 0.019222267; JUN/JUN-FOS_(complex): −0.019184424; MYC/Max_(complex): −0.018553276; XBP1-2: −0.017009915; negative_regulation_of_DNA_binding_(abstract): −0.016224139; PPARGC1A: −0.015525361; p53_tetramer_(complex): −0.013881353; TP63-5: 0.011860936; p53_(tetramer)_(complex): −0.011120564; FOXM1: 0.010515289; MIR146A_(miRNA) −0.004588203; MIR200A_(miRNA): 0.004570842; MIR22_(miRNA): −0.00455296; MIRLET7G_(miRNA): −0.004534414; MIR26A1_(miRNA): −0.004515057; MIR141_(miRNA): 0.004494806; MIR338_(miRNA): 0.004473776; MIR23B_(miRNA): −0.004452502: MIR9-3_(miRNA): 0.004432174; MIR26B_(miRNA): −0.004414627; MIR429_(miRNA): 0.004401701; MIR26A2_(miRNA): −0.004393525; MIR17_(miRNA): 0.004385947; DLEU2_(rna): −0.004376141; DLEU1_(rna): −0.004337657; TP53: −0.003302879; JUN: 0.003189085; NOTCH4_(rna): 0.002218066; and E2F1/DP_(complex): 0.000376653.

In still further contemplated aspects, the inventors also contemplate the use of a plurality of entity coefficients of a high-accuracy gain response predictor to modify output of a pathway model to so predict a treatment outcome for a patient, wherein the high-accuracy gain response predictor is associated with a drug, and wherein the pathway model uses omics data of the patient.

Most typically, the plurality of entity coefficients is between one and 50 entity coefficients of the high-accuracy gain response predictor, and the plurality of entity coefficients is a subset of entity coefficients and comprises the top tertile of all entity coefficients of the high-accuracy gain response predictor. As noted before, it is generally preferred that the pathway model is a probabilistic pathway model (e.g., PARADIGM), and that the drug is a chemotherapeutic drug.

Various objects, features, aspects and advantages of the inventive subject matter will become more apparent from the following detailed description of preferred embodiments, along with the accompanying drawing figures in which like numerals represent like components.

### BRIEF DESCRIPTION OF THE DRAWING

FIGS. 1A-1C schematically illustrate exemplary aspects of response predictors contemplated herein.

FIG. 2 exemplarily and schematically illustrates a process according to the inventive subject matter.

FIG. 3 exemplarily illustrates a ranked listing of calculated treatment responses/test models in which responses/models with higher accuracy gain over null models are placed to the left of those with lower accuracy gain. The calculated treatment response/test model at the far left predicted sensitivity of the patient to dasatinib with the highest accuracy gain.

FIG. 4 depicts exemplary results of accuracy gains for different calculations using different pathway models and omics input.

FIG. 5 is an exemplary representation of dasatinib sensitivity sorted by human TCGA tumor tissue type.

FIG. 6 is an exemplary representation of dasatinib sensitivity sorted by specific human TCGA tumors.

### DETAILED DESCRIPTION

The inventor has discovered that generation of a large quantity of response predictors from pathway model analyses are not only useful in the identification of high-accuracy models but can also be used to obtain entity coefficients useful for prediction of treatment outcome for a patient based on the patient's specific omics data. Viewed from a different perspective, it should be appreciated that machine learning on pathway analyses for multiple experimental, curated, and/or actual treatment data (e.g., for a variety of drugs and conditions with known outcome relative to a drug treatment and a disease and with known omics data) will provide response prediction models that in turn provide entity coefficients associating a specific treatment outcome with a specific drug. These entity coefficients can then be used as factors for a pathway model output based on actual patient omics data to so predict a likely treatment outcome where the patient is treated with that drug.

In one example, as further described in more detail below, the inventor first obtained a relatively large number of genome-wide assays (typically including RNA expression levels, DNA sequence information and copy-number information), totaling about 1,000 cell lines derived from multiple tissue types. Inferred pathway activities (IPAs) were then generated based on expression and copy-number data using PARADIGM software. In a still further step, the inventor also obtained drug response data (GI₅₀) for approximately 140 compounds in these cell lines, and multiple cross-validated response predictors were built for each compound in Topmodel software. Notably, it was discovered that for the cell lines tested, dasatinib was the most accurately predicted drug response by observing cross-validated accuracies in multiple models, and the top dasatinib response prediction model was then further analyzed. In one analysis, as is also shown in more detail below, the top dasatinib response prediction model was demonstrated to have predictive utility in nervous system cell types, which was also validated by findings when the top response prediction model was tested against primary cancer patient data (TCGA). Notably, dasatinib is an approved drug for treatment of acute lymphoblastic leukemia. It should therefore be appreciated that contemplated systems and methods allow prediction of a treatment outcome for treatment with a drug in a condition for which use of that drug is not known or approved. Moreover, it is noted that the entity coefficients of the so identified response prediction model can then be used to predict treatment outcome for a patient using the patient's actual omics data.

In this context, it should be appreciated that an overwhelming amount of machine learned predictive models can be prepared that allow calculation of a prediction (e.g., sensitivity) score on the basis of various omics datasets and/or pathway models prepared from omics datasets. Unfortunately, all of these models have various inherent biases, for example, due to underlying mathematical assumptions in machine learning and pathway construction, use of specific cell cultures or biopsy samples to obtain the omics data, the drug used with the cell cultures or biopsy samples, etc. Nevertheless, all of these models are based on actual cell biological processes and therefore provide at least potentially valuable insights. However, none of the diverse models provides any guidance as to which model will provide a match to a particular patient omics sample or pathway model that would predict whether or not a particular drug is likely to have a desired treatment outcome in the patient.

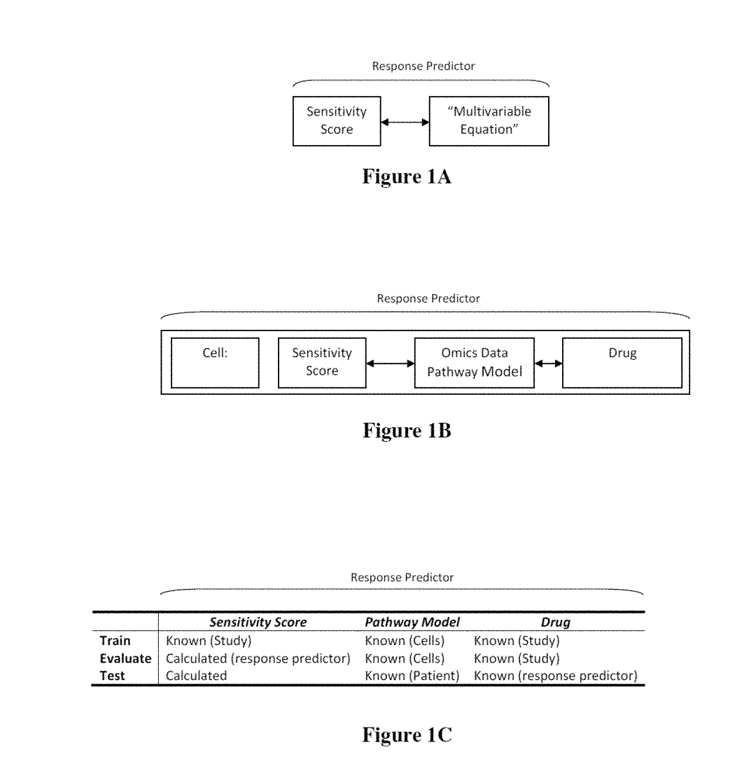

The inventors have now discovered systems and methods for matching actual patient data, and particularly pathway models from data of a patient, with a drug-specific response predictor that has a desirably high gain of accuracy over a corresponding null model, which in turn allows calculation of a likely treatment outcome of that patient using the specific drug. In that context, as simplified in FIG. 1A, an exemplary response predictor (predictive model) can be viewed as multivariable equation obtained from a machine learning algorithm that will give a sensitivity or prediction score. More particularly, and as further exemplarily illustrated in FIG. 1B, a response predictor is generated using a machine learning algorithm that uses omics data and/or pathway models generated from a cell culture or tissue exposed to a drug. As is indicated in FIG. 1B, cells or tissue are exposed to a drug and sensitivity is observed (e.g., quantified as IC₅₀, EC₅₀, etc., or qualitatively assessed as sensitive or resistant), most typically in comparison with a negative or otherwise contrasting control (e.g., without drug or with different cell type). Omics data and/or pathway models from the cells/tissue are then used in a machine learning algorithm together with the observed factors as training data to so arrive at a response predictor. Of course, it should be appreciated that the same omics data and/or pathway models and observed factors can be used as training data in more than one machine learning algorithm, and it should be appreciated that all known machine learning algorithms are deemed suitable for use herein. Consequently, it should be appreciated that one set of in vitro experiments can provide a multiplicity of trained models (i.e., response predictors generated by respective machine learning algorithms). As is also well known in the art, available data may be split into a training set and evaluation set to obtain trained models, or all data can be used to get a fully trained model. Viewed from a different perspective, and as schematically shown in FIG. 1C, a response predictor can be generated using machine learning algorithms using training data where sensitivity of a cell or tissue to a drug is known, where the drug is known, and where the omics data and/or pathway model is readily obtained from the cells or tissue. So generated trained models can then be validated using evaluation data which can be from the same dataset as the training data, and as before, the sensitivity of a cell or tissue to the drug is known, the drug is known, and the omics data and/or pathway model are readily obtained from the cells or tissue. Thus, it should be appreciated that numerous in vitro tests will form the basis for a large variety of response predictors that can then be used for calculation with a patient's omics data or pathway models. Using the patient omics data or pathway models in conjunction with the response predictors will then provide a predicted response score (predicted treatment outcome, or predicted sensitivity) for a drug.

Most advantageously, it should be recognized that contemplated systems and methods take advantage of the growing number of omics information associated with drugs and cells or tissue types. Moreover, while the examples presented herein were based on multiple and distinct drugs and cell lines, it should be appreciated that response predictors can be built from omics data of cells, curated data, and treatment data related only to a single drug (typically in conjunction with a plurality of distinct diseased (e.g., cancer) cell lines with distinct response profiles). Regardless of the particular drug(s) investigated, and using such information, a vast number of individual response predictors can be prepared, and it should therefore be recognized that the collection of response predictors need not be limited to a specific cancer type and/or therapeutic drug. For example, as is further explained in more detail below, the inventors obtained different omics data sets from publically available sources (e.g., CCLE expression, CCLE copy number, sanger expression, sanger copy number) as pathway model omics data, and also used the same omics data in a factor-graph-based pathway model (here: PARADIGM) to end up with 10 different input data collections for which 139 different drugs were reported. These pathway models and known drug responses were then subjected to 13 different machine learning algorithms (Linear kernel SVM, First order polynomial kernel SVM, Second order polynomial kernel SVM, Ridge regression, Lasso, Elastic net, Sequential minimal optimization, Random forest, J48 trees, Naive bayes, JRip rules, HyperPipes, and NMFpredictor) resulting in a total of 176,112 response predictors.

In this context it must be noted that each type of response predictor includes inherent biases or assumptions, which may influence how a resulting response predictor would operate relative to other types of response predictors, even when trained on identical data. Accordingly, different response predictors will produce different predictions/accuracy gains when using the same training data set. Heretofore, in an attempt to improve prediction outcome, single machine learning algorithms were optimized to increase correct prediction on the same data set. However, due to inherent bias of the algorithms, such optimization will not necessarily increase accuracy (i.e., accurate prediction capability against ‘coin flip’) in predictability. Such bias can be overcome by training numerous diverse response predictors with different underlying principles and classifiers on disease-specific data sets with associated metadata and by selecting from the so trained response predictors those with desirable prediction power over the corresponding null model.

Of course, it should be appreciated that the above is only an exemplary scenario with a relatively limited set of data, and that numerous additional data (e.g., in vitro data, clinical trial data, research data, treatment data, etc.) can be employed, each in combination with their respective drugs, and each calculated with different machine learning algorithms to so arrive at very large numbers (e.g., between 100,000-500,000, or between 500,000 and 1,000,000, or between 1,000,000 and 5,000,000, or between 5,000,000 and 10,000,000, and even more) of individual response predictors. As should be evident, such calculations well exceed multiple lifetimes of a human without computing infrastructure.

As should also be readily appreciated, even with computing infrastructure, such large data quantities would require immense computational effort where an actual dataset (omics data or pathway model) of a patient should be aligned with a dataset of a cell or tissue culture. The inventors have now discovered that even massive collections of response predictors can be effectively and expeditiously analyzed in a conceptually simple manner by calculating two predicted responses for a single response predictor, using a simulated null set and an actual patient dataset (omics data or pathway model). Differences between the predicted responses are then used to evaluate the performance of any single response predictor. In that manner, only relatively simple calculations are required and can be performed in a comparably small amount of time as the response predictors are relatively simple.

Consequently, it should be noted that the inventive subject matter presented herein enables construction or configuration of a computing device(s) to operate on vast quantities of digital data, beyond the capabilities of a human. Although the digital data can represent machine-trained computer models of omics data and treatment outcomes, it should be appreciated that the digital data is a representation of one or more digital models of such real-world items, not the actual items. Rather, by properly configuring or programming the devices as disclosed herein, through the instantiation of such digital models in the memory of the computing devices, the computing devices are able to manage the digital data or models in a manner that would be beyond the capability of a human. Furthermore, the computing devices lack a priori capabilities without such configuration. In addition, it should be appreciated that the present inventive subject matter significantly improves/alleviates problems inherent to computational analysis of complex omics calculations, provides guidance as to the proper model selection and eliminates bias due to an a priori selected machine learning algorithm.

Viewed from a different perspective, it should be appreciated that the present systems and methods in computer technology are used to solve a problem inherent in computing models for omics data. Thus, without computers, the problem, and thus the present inventive subject matter, would not exist. More specifically, systems and methods presented herein result in one or more drug-specific response predictors models having greater accuracy gain than others, which provide entity coefficients for rapid determination of treatment outcome prediction, leading ultimately to less latency in generating predictive results based on actual patient data.

It should be noted that any language directed to a computer, analysis engine, or machine learning system should be read to include any suitable combination of computing devices, including servers, interfaces, systems, databases, agents, peers, engines, controllers, modules, or other types of computing devices operating individually or collectively. One should appreciate the computing devices comprise a processor configured to execute software instructions stored on a tangible, non-transitory computer readable storage medium (e.g., hard drive, FPGA, PLA, solid state drive, RAM, flash, ROM, etc.). The software instructions configure or otherwise program the computing device to provide the roles, responsibilities, or other functionality as discussed below with respect to the disclosed apparatus. Further, the disclosed technologies can be embodied as a computer program product that includes a non-transitory computer readable medium storing the software instructions that causes a processor to execute the disclosed steps associated with implementations of computer-based algorithms, processes, methods, or other instructions. In some embodiments, the various servers, systems, databases, or interfaces exchange data using standardized protocols or algorithms, possibly based on HTTP, HTTPS, AES, public-private key exchanges, web service APIs, known financial transaction protocols, or other electronic information exchanging methods. Data exchanges among devices can be conducted over a packet-switched network, the Internet, LAN, WAN, VPN, or other type of packet switched network, circuit switched network, and/or cell switched network.

As used in the description herein and throughout the claims that follow, when a system, engine, server, device, module, or other computing element is described as configured to perform or execute functions on data in a memory, the meaning of “configured to” or “programmed to” is defined as one or more processors or cores of the computing element being programmed by a set of software instructions stored in the memory of the computing element to execute the set of functions or operate on target data or data objects stored in the memory.

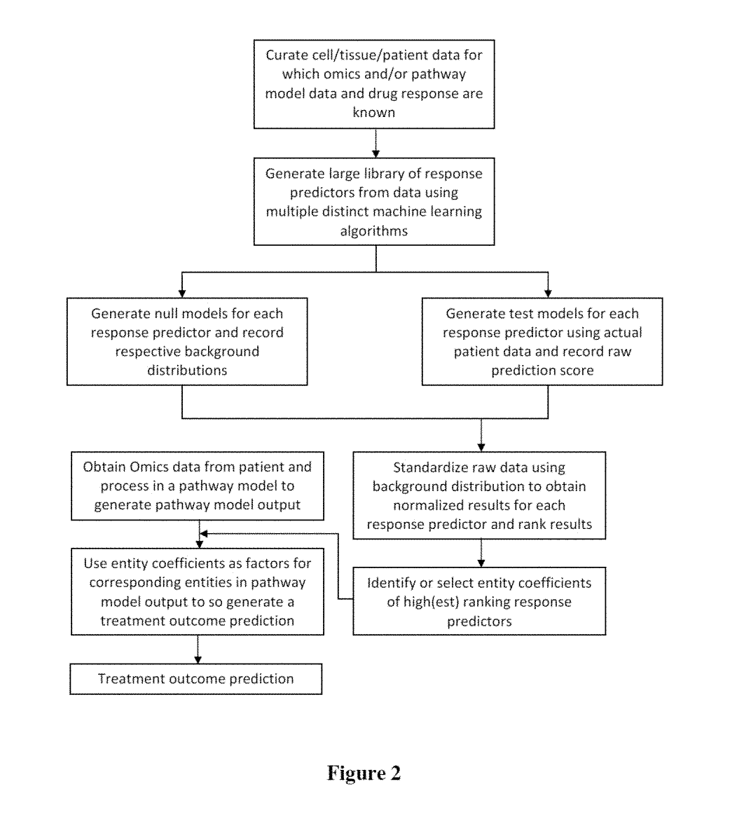

The flow chart of FIG. 2 exemplarily illustrates a typical workflow according to the inventive subject matter. Here, in a first step, a plurality of cell/tissue/patient data for which omics and/or pathway model data and drug responses are known are curated. Of course, it should be appreciated that all known forms of information suitable for the curation of such data are deemed suitable for use herein and include patient data from a medical service provider, lab, hospital, academic institution, and/or insurance carrier. Therefore, the data may be printed or in electronic format from a database or analytic device. Moreover, it should be appreciated that the data need not necessarily be derived from human studies, but may also be of non-human origin (e.g., rodent, simian, etc.). Likewise, the data may be derived from cell or tissue cultures. Additionally, where the data are raw or omics data, such data will typically be processed in a pathway analysis system, and particularly preferred pathway model systems include factor graph-based systems (e.g., PARADIGM). Still further, it is generally preferred that the data also include information about a drug or drugs used to treat the cells, tissue, or patient, as well as an appropriate outcome descriptor (e.g., drug sensitivity for cells or tissues, or partial or complete response, disease free survival, relapse, remission for human).

In one contemplated example, initial data may be curated from a collection of distinct cancer cell lines of a specific cancer cell type (e.g., melanoma) with known sensitivity to a specific drug for each of the cell lines. Such sensitivity may be experimentally determined, or curated form the literature. Alternatively or additionally, instead of using a collection of distinct cancer cell lines of a specific cancer cell type, the data may be curated from biopsy samples of a specific cancer cell type, and sensitivity to a drug may be determined in vitro, or inferred from patient treatment outcome where the patient was subjected to treatment with the drug. In another contemplated example, the data may be curated from published sources (e.g., clinical trials, scientific papers, annotated omics databases, etc.) where the omics data are available for cells or tissues with known sensitivity to a specific drug. In further examples, it should be appreciated that the cells or tissues need not necessarily be from the same cancer type, but indeed may originate from multiple and distinct cancer types (e.g., cancers of the nervous system, cancers of the lung, digestive system, urogenital system, skin, kidney, breast, thyroid, blood, bone, pancreas, soft tissue, etc) Likewise, it should be appreciated that the known sensitivity of the cells (of the same cancer type or of multiple cancer types) need not be limited to a single drug, but that multiple drug sensitivities may be used in the same analysis. Viewed from a different perspective, use of multiple cell lines/tissue/biopsy samples with known sensitivity or other outcome predictor may be employed as input data to generate a plurality of distinct response predictors.

Most typically, and depending on the source of initial data, the data will be omics data such as whole genome sequencing data, exome sequencing data, RNA sequencing and/or transcription level data, quantitative proteomics data, and/or protein activity data. Preferably, these data are then processed to obtain pathway activity information, and all known pathway analysis methods and algorithms are deemed suitable for use herein, including GSEA, SPIA, PathOlogist, ARACNE, MINDy, CONEXIC, NetBox, and MEMo. However, in especially preferred aspects, pathway analysis is performed using PARADIGM, which is a factor graph framework for pathway inference on high-throughput genomic data. Here, a gene is modeled by a factor graph as a set of interconnected variables encoding the expression and known activity of a gene and its products, allowing the incorporation of many types of omic data as evidence. Such method allows for prediction of the degree to which a pathway's activities (e.g., internal gene states, interactions or high-level ‘outputs’) are altered in a patient using probabilistic inference (see e.g., Bioinformatics. 2010 Jun. 15; 26(12): i237-i245). It should also be noted that pathway analysis on omics data advantageously and substantially reduces the volume of data that would otherwise be processed via machine learning. Instead, pathway analysis (especially where PARADIGM is employed) provides a relatively simple data structure in which a pathway element (e.g., gene, protein, protein complex) is associated with a numeric factor or value.

Using this information (e.g., drug response and pathway model for the specific cells or tissues, typically in conjunction with negative control and/or other parameter or metadata), a response predictor can then be calculated using a specific machine learning algorithm. In most preferred aspects, however, numerous additional response predictors are generated on the same information using multiple distinct other machine learning algorithms to so obtain a library of distinct response predictors. As already noted above, additional different drugs, omics datasets, pathway modeling, and cell types can additionally be used with additional multiple different machine learning algorithms, which will exponentially increase the number of available response predictors. Indeed, using such combinatorics, it should be recognized that the number of response predictors, even for a single drug, can readily exceed 1,000, more typically at least 10,000, even more typically at least 100,000 response predictors, all of which can then be collected into a response predictor library. However, it should be recognized that a response predictor is relatively simple and has a small data/file size as is exemplarily shown in FIG. 1A. In essence, a response predictor can be viewed as a multi-variable equation that comprises multiple pathway elements and associated factors and that so allows a simple calculation of a sensitivity (or other outcome measure) score using measured omics data of a cell or biopsy.

Once the response predictors are created, prediction quality for each of the response predictors may be assessed, and most preferably response predictors are retained that have a prediction power that exceeds random selection. Viewed from a different perspective, the various response prediction models may be assessed on their gain in accuracy. As will be readily appreciated, there are numerous manners of assessing accuracy, and the particular choice may depend at least in part on the metrics and algorithms used. For example, suitable metrics include an accuracy value, an accuracy gain, a performance metric, or other measure of the corresponding model.

Additional example metrics include an area under curve metric, an R² value, a p-value metric, a silhouette coefficient, a confusion matrix, or other metric that relates to the nature of the response predictor. Depending on the number of response predictors or accuracy distribution, it should be appreciated that a response predictor used for prediction may be selected as being the top model (e.g., having highest accuracy gain, or highest accuracy score, etc.), or as being in the top n-tile (tertile, quartile, quintile, etc.), or as being in the top n % of all models (top 5%, top 10%, etc.). For example, high accuracy gain models will typically be in the top quartile of accuracy gain.

The library of response predictors or individual response predictors (both are typically selected using a minimum prediction power exceeding random selection as noted above) may then be used for statistical selection of matches with a high prediction score for actual patient data using null models for each of the response predictors in the database. More specifically, null models are calculated for each of the response predictors using a moderate number (e.g., 100-500, or 500 to 1,000, or 1,000 to 10,000) of randomly chosen datasets. Most typically these data sets include pathway model data and/or omics data used in the calculation of the response predictors, but not used in calculation of the response predictor for which the null model is created. As can be expected, the so calculated null models provide a background signal distribution (e.g., mean and standard deviation) for unrelated or poorly-matched pathway models or omics data, that can be used for further normalization and ranking of results.

For example, in situations where one response predictor predicts a high prediction score (e.g., high level of sensitivity or resistance) for a known data set and known outcome and an average prediction score for the randomly chosen datasets (background signal), a high score is noted as the raw score that is then adjusted using the background signal distribution to so arrive at a standardized score. It should be appreciated that this standardized score characterizes the conformance of the known data set with the performance of the response predictor as originally calculated with the drug of a particular cell or tissue. Thus, a comparison between the null model and corresponding test model or top model (model with highest accuracy gain among corresponding models), and the difference in raw score, and more preferably the difference in standardized score can be used for ranking. Top ranking response predictors (for each drug, where multiple drugs were tested) are identified, along with the pathway entities and associated entity coefficients. So selected response predictor(s) can then be used in various manners, and especially for prediction of treatment response to a drug based on actual patient omics and pathway analysis data. Thus, and unless indicated otherwise, the term “high-accuracy gain response predictor” as used herein refers to a response predictor that has a ranking in the top tertile in a standardized ranking of response predictors.

As noted above, it should be particularly appreciated that each response predictor will have a relatively simple data structure and enumerates a plurality of entity designators (e.g., pathway entities such as MIR34A, AP1 complex, TP63, etc.) along with the corresponding entity coefficients (typically a numeric value). Where desired, the function of the entity (e.g., cell cycle, apoptosis, etc.; unknown function is denoted as NULL) may also be included as is exemplarily shown for a response predictor in Table 1 below.

TABLE 1

| Entity/PARADIGM label | Coefficient | Function |
|---|---|---|
| MIR34A_(miRNA) | −0.10545895 | NULL |
| ETS1 | −0.094264817 | NULL |
| 5_8_S_rRNA_(rna) | 0.086044958 | NULL |
| CEBPB_(dimer)_(complex) | 0.067691407 | Immune signaling |
| FOSL1 | −0.067263561 | JUN/FOS Family |
| CEBPB | 0.066698569 | Immune signaling |
| JUN/FOS_(complex) | −0.064549881 | JUN/FOS Family |
| Fra1/JUN_(complex) | −0.060403293 | JUN/FOS Family |
| FOXA2 | 0.059755319 | Differentiation |
| FOS | −0.059560833 | JUN/FOS Family |
| E2F1 | −0.050992273 | Proliferation |
| AP1_(complex) | −0.049823492 | JUN/FOS Family |
| anoikis_(abstract) | −0.04853399 | Apoptosis |
| FOXA1 | 0.035994367 | Differentiation |
| dNp63a_(tetramer)_(complex) | −0.033478521 | Cell-cycle checkpoint |
| TP63 | −0.02956134 | Cell-cycle checkpoint |
| MYC | 0.026847479 | Apoptosis |
| TP63-2 | −0.026423542 | Cell-cycle checkpoint |
| E2F-1/DP-1_(complex) | −0.023462081 | Proliferation |
| MYB | 0.022211938 | Proliferation |
| TAp63g_(tetramer)_(complex) | 0.019789929 | Cell-cycle checkpoint |
| HIF1A/ARNT_(complex) | 0.019222267 | Angiogenesis |
| JUN/JUN−FOS_(complex) | −0.019184424 | JUN/FOS Family |
| MYC/Max_(complex) | −0.018553276 | Apoptosis |
| XBP1-2 | −0.017009915 | Immune signaling |
| negative_regulation_of_DNA_binding_(abstract) | −0.016224139 | Cell-cycle checkpoint |
| PPARGC1A | −0.015525361 | NULL |
| p53_tetramer_(complex) | −0.013881353 | Cell-cycle checkpoint |
| TP63-5 | 0.011860936 | Cell-cycle checkpoint |
| p53_(tetramer)_(complex) | −0.011120564 | Cell-cycle checkpoint |
| FOXM1 | 0.010515289 | Cell-cycle checkpoint |
| MIR146A_(miRNA) | −0.004588203 | NULL |
| MIR200A_(miRNA) | 0.004570842 | NULL |
| MIR22_(miRNA) | −0.00455296 | NULL |
| MIRLET7G_(miRNA) | −0.004534414 | NULL |
| MIR26A1_(miRNA) | −0.004515057 | NULL |
| MIR141_(miRNA) | 0.004494806 | NULL |
| MIR338_(miRNA) | 0.004473776 | NULL |
| MIR23B_(miRNA) | −0.004452502 | NULL |
| MIR9-3_miRNA) | 0.004432174 | NULL |
| MIR26B_(miRNA) | −0.004414627 | NULL |
| MIR429_(miRNA) | 0.004401701 | NULL |
| MIR26A2_(miRNA) | −0.004393525 | NULL |
| MIR17_(miRNA) | 0.004385947 | NULL |
| DLEU2_(rna) | −0.004376141 | Tumor-suppressor |
| DLEU1_(rna) | −0.004337657 | Tumor-suppressor |
| TP53 | −0.003302879 | Cell-cycle checkpoint |
| JUN | 0.003189085 | JUN/FOS Family |
| NOTCH4_(rna) | 0.002218066 | Angiogenesis |
| E2F1/DP_(complex) | 0.000376653 | Proliferation |

Using the response predictors, it should be recognized that patient data obtained from a pathway model output of an actual patient can be processed using entity coefficients for corresponding pathway entities in the response predictors. For example, where the pathway model output (based on patient omics data) for a first pathway entity (e.g., AP1) is a first value, that first value can be modified by the corresponding coefficient (e.g., coefficient for AP1) in the response predictor to so produce a first modified value, etc. The totality of modified output entity values (modified by the corresponding coefficients) will then provide a numeric indication that corresponds to the models calculated sensitivity (or other outcome measure) score, which corresponds to a calculated prediction for a treatment outcome (e.g., positive numeric value for drug sensitivity).

In further contemplated aspects, it should also be appreciated that the systems and methods presented herein may also be used to identify one or more pharmaceutical agents (e.g., investigational drugs or drug candidates in a development pipeline where multiple cell lines are exposed to multiple investigational drugs or drug candidates) with a desirably high degree of accuracy for response prediction. Such identification is especially beneficial where multiple drugs are under development and where contemplated systems and methods identify a drug as having a sensitivity (or other outcome measure) score that can be predicted with a desirably high degree of accuracy. Still further, contemplated systems and methods are also suitable to identify a drug in an indication that not been previously recognized or appreciated as is shown in more detail below. In short, contemplated systems and methods may be used where multiple drugs for multiple indications are tested. The response prediction models are finally ranked according to the highest accuracy gain per drug, and then by drug (with the highest accuracy gain).

It should be especially appreciated that such calculation is rapid due to the simplified data structure of the response predictors and will not require a machine learning process in which patient data are attempted to conform to in vitro model data as would be commonly done.

### Examples

Based on various omics data (e.g., transcription and copy number) and pathway data (e.g., PARADIGM) from patients diagnosed with glioblastoma, and response predictors built from known genomic datasets of different cell types, exposure to different drugs, and the respective associated sensitivities to the drugs, in combination with various different machine learning classifiers as shown in Table 2 below, dasatinib was identified as a drug suitable for the patients diagnosed with glioblastoma.

TABLE 2

| Types | | Number |
|---|---|---|
| Genomic datasets | CCLE expression | 10 (8320 samples) |
| CCLE copy number | | |
| CCLE expression paradigm | | |
| CCLE copy | | |
| number paradigm | | |
| CCLE expression | | |
| & copy number | | |
| paradigm | | |
| sanger expression | | |
| sanger copy number | | |
| sanger expression | | |
| paradigm | | |
| sanger copy number | | |
| paradigm | | |
| sanger_expression | | |
| & copy number | | |
| paradigm | | |
| Drugs | 17-AAG | 139 |
| 681640 | | |
| A-443654 | | |
| A-770041 | | |
| . . . | | |
| WZ-1-84 | | |
| XMD8-85 | | |
| Z-LLNle-CHO | | |
| ZM-447439 | | |
| Classifiers | Linear kernel SVM | 13 |
| First order | | |
| polynomial kernel | | |
| SVM | | |
| Second order | | |
| polynomial kernel | | |
| SVM | | |
| Ridge regression | | |
| Lasso | | |
| Elastic net | | |
| Sequential minimal | | |
| optimization | | |
| Random forest | | |
| J48 trees | | |
| Naive bayes | | |
| JRip rules | | |
| HyperPipes | | |
| NMFpredictor | | |
| Feature selections | Four levels of | 4 |
| | variance filters | |

More specifically, using the above data sets, drugs, and classifiers, 29,352 fully trained drug response models were built, 146,760 additional evaluation models were built (at 5-fold CV), and 176,112 total models were analyzed, yielding a large number of response predictors for various drugs. Genomic-scale data from glioblastoma patients were collected from individual cancer samples via microarray or sequencing technology. Independent assays were performed on the same samples (e.g., expression profiling and copy-number estimation) to evaluate what data type will provide best predictions. These patient data were integrated in a factor-graph-based model (PARADIGM). The most likely state for the pathway networks given the omics data evidence was estimated, and reported as inferred pathway activities (i.e., a pathway model was established with activities for respective pathway elements). In this context, it should be especially appreciated that the contemplated systems and methods are neither based on prediction optimization of a singular model nor based on identification of best correlations of selected omics parameters with a treatment prediction.

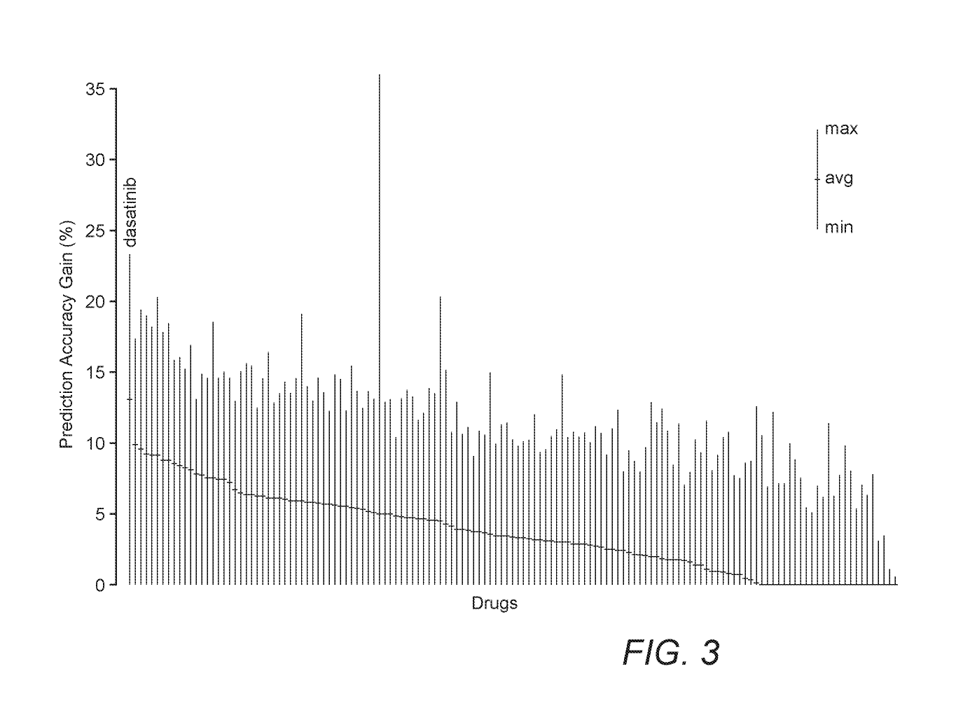

Using the response predictors in the predictor database and actual patient data, null models were then calculated for each of the response predictors with 1,000 randomly selected datasets, and mean and standard deviation were recorded for each null model. Test models were then calculated using patient datasets for each of the response predictors and the results were standardized using the results from the respective null models. FIG. 3 exemplarily shows ranking of standardized scores. Here, each vertical line represents average, minimum, and maximum results for a number of response predictors, grouped by a specific drug. As can be seen from FIG. 3, response predictors to the left are more consistently accurately predicted, and the most consistently predicted drug is dasatinib for the patients diagnosed with glioblastoma. Notably, it should be appreciated that dasatinib was originally developed as an oral Bcr-Abl tyrosine kinase inhibitor (inhibits the “Philadelphia chromosome” protein) and was approved for first line use in patients with chronic myelogenous leukemia and Philadelphia chromosome-positive acute lymphoblastic leukemia. Of course, it should also be appreciated that the above process can be modified to include as initial data only data from glioblastoma (or other selected cancer) using only different glioblastoma (or other selected cancer) cancer cell lines or biopsies, and using only a drug known or suspected to be effective in the treatment of glioblastoma. Such modified process will then yield response predictors that are specific to glioblastoma (or other selected cancer) and a specific drug only. On the other hand, the above process can be modified to include as initial data only data from glioblastoma (or other selected cancer) using only different glioblastoma (or other selected cancer) cancer cell lines or biopsies and multiple different drugs that are (optionally) known or suspected to be effective in the treatment of glioblastoma. Such modified process will then yield response predictors that are specific to glioblastoma (or other selected cancer) and multiple drug candidates.

Thus, it should be appreciated that a response to a drug in a patient can be predicted (a) in a manner that is agnostic of the drug target and (b) on the basis of omics data/pathway models of the patient when used as input data to a collection of prediction models where each of the models was optimized to predict drug response as a function of a specific set of omics data/pathway models. Moreover, by comparing predicted results to corresponding null models, statistically relevant predictions above background are reported, which then allows for ranking the response predictions. Additionally, to ensure that the patient data do not import an inherent bias, permutations can also be generated from the patient data that are then classified in a manner as described for the null models to ensure that the patient data and the null model are distributed similarly.

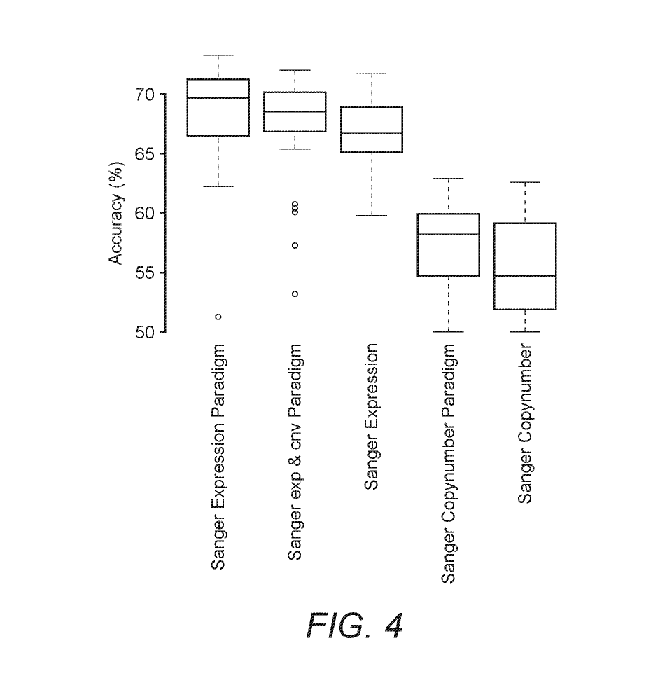

With respect to the omics data and pathway models suitable for use herein, it should be noted that all omics data and pathway models are deemed appropriate, and exemplary omics data include sequencing data, especially tumor versus normal data, such as whole genome sequencing data, exome sequencing date, etc. Moreover, suitable omics data also include transcriptomics data and proteomics data. Likewise, suitable pathway analyses include Gene Set Enrichment Analysis (GSEA, Broad Institute) based models, Signaling Pathway Impact Analysis (SPIA, Bioconductor) based models, and PathOlogist pathway models (NCBI) as well as factor-graph based models, and especially PARADIGM as described in WO2011/139345A2, WO2013/062505A1, and WO2014/059036, all incorporated by reference herein. FIG. 4 provides exemplary comparative results depicting average accuracy as a function of the type of omics data and pathway models. As can be clearly seen, the highest accuracy was achieved using Sanger expression data that were processed using PARADIGM to so obtain a pathway model. Similarly high accuracy was achieved using Sanger expression and copy number data, again processed using PARADIGM to so obtain the corresponding pathway model. Notably, Sanger expression data alone without pathway modeling also afforded relatively high, albeit somewhat lower, accuracy. Copy number omics data only, per se or processed using PARADIGM, ranked somewhat lower.

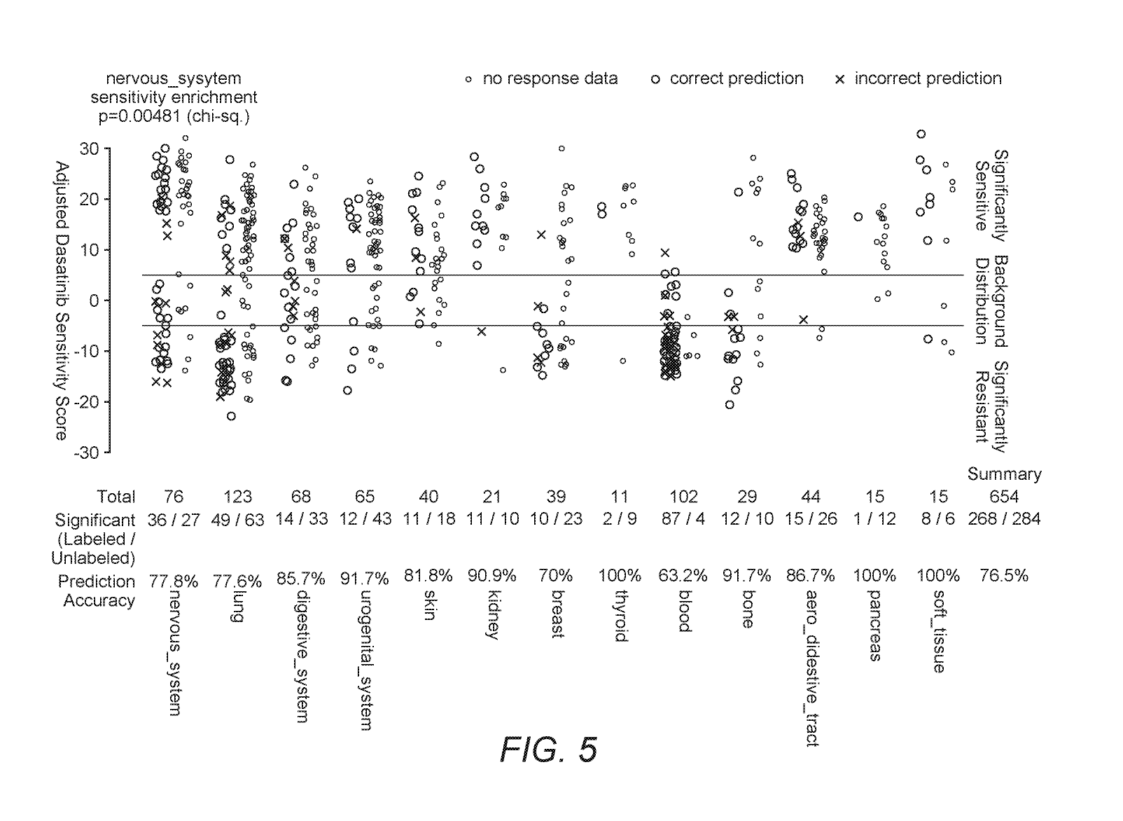

The accuracy of the so obtained predictions was also cross-checked using omics data and pathway models for cell lines, and the results are depicted in FIG. 5. Here, the adjusted sensitivity scores are plotted with solid circles indicating predictions for which sensitivity data were available, with empty circles indicating predictions for which sensitivity data were not available, and labeled with x for incorrect predictions. Notably, prediction accuracy for dasatinib in neural cell lines was 77.8%, which coincides with the prediction for glioblastoma patients.

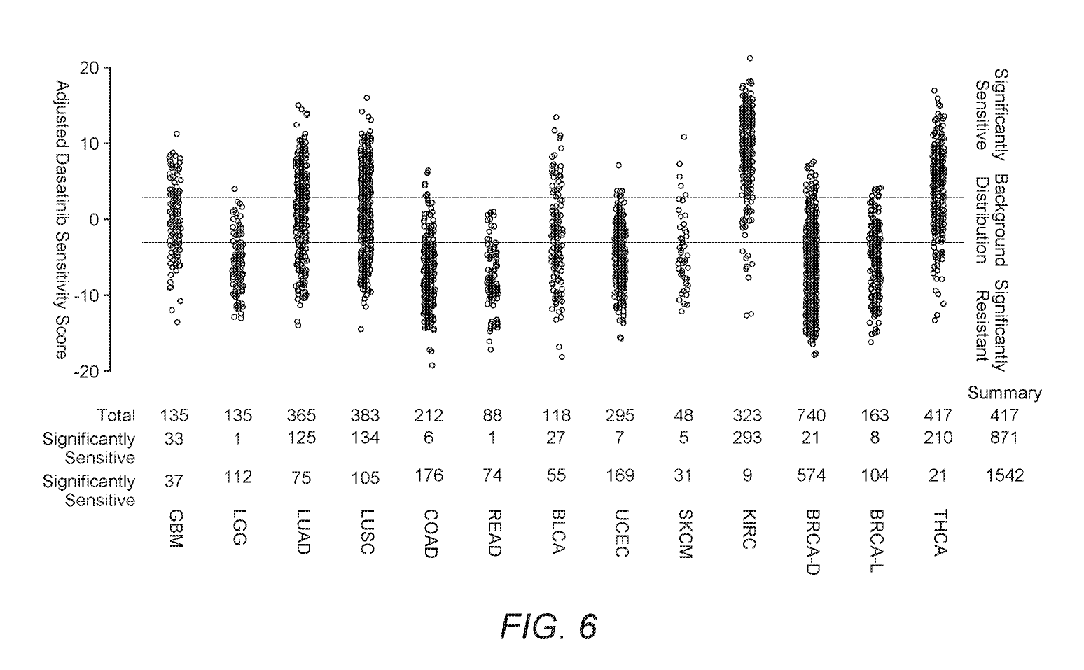

Equally notable is that dasatinib resistance can be accurately predicted as well as can be taken from FIG. 5. A similar cross check was performed using primary patient data from TCGA samples in tissues that correspond to the training cell line panel as can be seen from FIG. 6. Note that the tissue effects behave similarly between cell line and patient data. For example, similarly to neural system lines, GBM patient samples are predicted to contain responder and non-responder subsets. In addition, it should be noted that dasatinib may be an excellent alternate drug candidate for human renal clear cell carcinoma. Most typically, as it was shown that the response predictor is particularly accurate with respect to neural tumors, the patient data will be obtained from a patient diagnosed with a neural tumor (e.g., glioblastoma). To that end, the tumor may be biopsied and omics data may be determined for the tissue sample, preferably against a matched normal control. The omics data are then processed in PARADIGM (or other suitable pathway analysis software) to obtain a pathway model that comprises data for entities corresponding to the entities in the response predictor. The patient PARADIGM values are then applied to the corresponding entity coefficients and a result based on the response predictor entity coefficients and actual pathway data from the patient will indicate the treatment outcome associated with the response predictor.

With further reference to the entity coefficients of Table 1 above, it should be evident that some (and more preferably all) of the so obtained coefficients for the top-ranking (or otherwise desired) response predictor for dasatinib can be used in conjunctions with actual patient data. Thus, a response predictor for treatment of glioblastoma with dasatinib can include at least two, or at least three, or at least five, or at least seven, or at least ten of the following entities and optionally respective coefficients (here listed as entity:coefficient pairs): MIR34A_(miRNA): −0.10545895; ETS1: −0.094264817; 5_8 S_rRNA_(rna): 0.086044958; CEBPB_(dimer)_(complex): 0.067691407; FOSL1: −0.067263561; CEBPB: 0.066698569; JUN/FOS_(complex): −0.064549881; Fra1/JUN_(complex): −0.060403293; FOXA2: 0.059755319; FOS: −0.059560833; E2F1: −0.050992273; AP1_(complex): −0.049823492; anoikis_(abstract): −0.04853399; FOXA1: 0.035994367; dNp63a_(tetramer)_(complex): −0.033478521; TP63: −0.02956134; MYC: 0.026847479; TP63-2: −0.026423542; E2F-1/DP-1_(complex): −0.023462081; MYB: 0.022211938; TAp63g_(tetramer)_(complex): 0.019789929; HIF1A/ARNT_(complex): 0.019222267; JUN/JUN-FOS_(complex): −0.019184424; MYC/Max_(complex): −0.018553276; XBP1-2: −0.017009915; negative_regulation_of_DNA_binding_(abstract): −0.016224139; PPARGC1A: −0.015525361; p53_tetramer_(complex): −0.013881353; TP63-5: 0.011860936; p53_(tetramer)_(complex): −0.011120564; FOXM1: 0.010515289; MIR146A_(miRNA)−0.004588203; MIR200A_(miRNA): 0.004570842; MIR22_(miRNA): −0.00455296; MIRLET7G_(miRNA): −0.004534414; MIR26A1_(miRNA): −0.004515057; MIR141_(miRNA): 0.004494806; MIR338_(miRNA): 0.004473776; MIR23B_(miRNA): −0.004452502: MIR9-3_(miRNA): 0.004432174; MIR26B_(miRNA): −0.004414627; MIR429_(miRNA): 0.004401701; MIR26A2_(miRNA): −0.004393525; MIR17_(miRNA): 0.004385947; DLEU2_(rna): −0.004376141; DLEU1_(rna): −0.004337657; TP53: −0.003302879; JUN: 0.003189085; NOTCH4_(rna): 0.002218066; and E2F1/DP_(complex): 0.000376653.

Further considerations suitable for use herein are disclosed in WO 2014/193982, filed 28 May 2014, in WO/2016/118527, filed 19 Jan. 2016, in WO/2016/141214, filed 3 Mar. 2016, and in WO/2016/205377, filed 15 Jun. 2016, all incorporated by reference herein.

As used in the description herein and throughout the claims that follow, the meaning of “a,” “an,” and “the” includes plural reference unless the context clearly dictates otherwise. Also, as used in the description herein, the meaning of “in” includes “in” and “on” unless the context clearly dictates otherwise. As also used herein, and unless the context dictates otherwise, the term “coupled to” is intended to include both direct coupling (in which two elements that are coupled to each other contact each other) and indirect coupling (in which at least one additional element is located between the two elements). Therefore, the terms “coupled to” and “coupled with” are used synonymously. Finally, and unless the context dictates the contrary, all ranges set forth herein should be interpreted as being inclusive of their endpoints, and open-ended ranges should be interpreted to include commercially practical values. Similarly, all lists of values should be considered as inclusive of intermediate values unless the context indicates the contrary.

It should be apparent to those skilled in the art that many more modifications besides those already described are possible without departing from the inventive concepts herein. The inventive subject matter, therefore, is not to be restricted except in the scope of the appended claims. Moreover, in interpreting both the specification and the claims, all terms should be interpreted in the broadest possible manner consistent with the context. In particular, the terms “comprises” and “comprising” should be interpreted as referring to elements, components, or steps in a non-exclusive manner, indicating that the referenced elements, components, or steps may be present, or utilized, or combined with other elements, components, or steps that are not expressly referenced. Where the specification claims refers to at least one of something selected from the group consisting of A, B, C . . . and N, the text should be interpreted as requiring only one element from the group, not A plus N, or B plus N, etc.

What is claimed is:

1. A method of processing a plurality of response predictors, comprising:
providing a plurality of response predictors, wherein each of the response predictors is associated with a drug and has a plurality of pathway elements and associated entity coefficients;
calculating an accuracy gain metric for each of the response predictors relative to a corresponding null model to select a single response predictor; and
using at least a subset of pathway elements and associated entity coefficients of the selected response predictor and a pathway model output of a patient tumor to calculate a score.

2. The method of claim 1 wherein the plurality of response predictors is at least 10,000 response predictors.

3. The method of claim 1 wherein the pathway element for the entity coefficient is selected form the group consisting of a regulatory RNA, a immune signaling component, a cell differentiation factor, a cell proliferation factor, an apoptosis signaling component, an angiogenesis factor, and a cell cycle checkpoint component.

4. The method of claim 1 wherein the accuracy gain metric is selected from the group consisting of an accuracy value, an accuracy gain, a performance metric, an area under curve metric, an R² value, a p-value metric, a silhouette coefficient, and a confusion matrix.

5. The method of claim 1 wherein the plurality of response predictors are established using at least two different machine learning classifiers.

6. The method of claim 6 wherein the at least two different machine learning classifiers are selected from the group consisting of a linear kernel support vector machine, a first or second order polynomial kernel support vector machine, a ridge regression, an elastic net algorithm, a sequential minimal optimization algorithm, a random forest algorithm, a naive Bayes algorithm, and a NMF predictor algorithm.

7. The method of claim 1 wherein the corresponding null model is calculated using randomly chosen datasets not used in calculation of the response predictor for which the null model is created.

8. A method of using an output of a pathway model of a tumor in a patient for prediction of a treatment outcome of the patient using a drug, comprising:
using a plurality entity coefficients of pathway elements in a high-accuracy gain response predictor for a drug as factors for output values of corresponding pathway elements in the pathway model of the tumor to predict a treatment outcome score for the patient using the drug;
wherein the pathway model of the tumor is calculated using omics data of the patient and comprises a plurality of pathway elements and associated output values;
wherein the high-accuracy gain response predictor has a predetermined minimum accuracy gain relative to a corresponding null model; and
wherein the high-accuracy gain response predictor is selected from a plurality of response predictors, wherein each of the response predictors is associated with the drug.

9. The method of claim 8 wherein the plurality of entity coefficients is a subset of entity coefficients and comprises the top tertile of all entity coefficients of the high-accuracy gain response predictor.

10. The method of claim 8 wherein the pathway model is PARADIGM.

11. The method of claim 8 wherein the predetermined minimum accuracy gain is at least 50% over the null model.

12. The method of claim 8 wherein the null model is calculated using randomly chosen datasets not used in calculation of the high-accuracy gain response predictor for which the null model is created.

13. The method of claim 8 wherein the plurality of response predictors are established using at least two different machine learning classifiers.

14. The method of claim 13 wherein the at least two different machine learning classifiers are selected from the group consisting of a linear kernel support vector machine, a first or second order polynomial kernel support vector machine, a ridge regression, an elastic net algorithm, a sequential minimal optimization algorithm, a random forest algorithm, a naive Bayes algorithm, and a NMF predictor algorithm.

15. The method of claim 8 wherein the drug is a chemotherapeutic drug.

16. A method of predicting a treatment outcome for treatment of a tumor of a patient with dasatinib, comprising:
obtaining omics data of the tumor of the patient;
calculating by a pathway analysis engine that uses a pathway model and the omics data, a pathway model output for the tumor, wherein the pathway output comprises a plurality of pathway elements and associated activity values;
applying a plurality of entity coefficients of respective pathway entities as factors to the activity values of corresponding pathway elements of the pathway model output to thereby predict the treatment outcome for the patient; and
wherein the pathway entities and respective entity coefficients are selected from the group consisting of MIR34A_(miRNA): −0.10545895; ETS1: −0.094264817; 5_8_S_rRNA_(rna): 0.086044958; CEBPB_(dimer)_(complex): 0.067691407; FOSL1: −0.067263561; CEBPB: 0.066698569; JUN/FOS_(complex): −0.064549881; Fra1/JUN_(complex): −0.060403293; FOXA2: 0.059755319; FOS: −0.059560833; E2F1: −0.050992273; AP1_(complex): −0.049823492; anoikis_(abstract): −0.04853399; FOXA1: 0.035994367; dNp63a_(tetramer)_(complex): −0.033478521; TP63: −0.02956134; MYC: 0.026847479; TP63-2: −0.026423542; E2F-1/DP-1_(complex): −0.023462081; MYB: 0.022211938; TAp63g_(tetramer)_(complex): 0.019789929; HIF1A/ARNT_(complex): 0.019222267; JUN/JUN-FOS_(complex): −0.019184424; MYC/Max_(complex): −0.018553276; XBP1-2: −0.017009915; negative_regulation_of_DNA_binding_(abstract): −0.016224139; PPARGC1A: −0.015525361; p53_tetramer_(complex): −0.013881353; TP63-5: 0.011860936; p53_(tetramer)_(complex): −0.011120564; FOXM1: 0.010515289; MIR146A_(miRNA) −0.004588203; MIR200A_(miRNA): 0.004570842; MIR22_(miRNA): −0.00455296; MIRLET7G_(miRNA): −0.004534414; MIR26A1_(miRNA): −0.004515057; MIR141_(miRNA): 0.004494806; MIR338_(miRNA): 0.004473776; MIR23B_(miRNA): −0.004452502: MIR9-3_(miRNA): 0.004432174; MIR26B_(miRNA): −0.004414627; MIR429_(miRNA): 0.004401701; MIR26A2_(miRNA): −0.004393525; MIR17_(miRNA): 0.004385947; DLEU2_(rna): −0.004376141; DLEU1_(rna): −0.004337657; TP53: −0.003302879; JUN: 0.003189085; NOTCH4_(rna): 0.002218066; and E2F1/DP_(complex): 0.000376653.

17. The method of claim 16 wherein the pathway model is a probabilistic pathway model.

18. The method of claim 16 wherein the pathway model is PARADIGM.

19. The method of claim 16 wherein the omics data of the patient comprise at least one of copy number data, expression level data, DNA sequence data, and mutation data.

20. The method of claim 16 wherein the tumor is a neural tumor.
